(19) United States
(12) Patent Application Publication
(10) Pub. No.: US 2012/0027859 A1
(43) Pub. Date: Feb. 2, 2012

(54) Biodegradable Proline-Based Polymers

(76) Inventors: William G. Turnell, Del Mar, CA (US); Zaza D. Gomurashvili, La Jolla, CA (US); Jeffrey Neil Anderl, San Diego, CA (US)

(21) Appl. No.: 13/062,690

(22) PCT Filed: Oct. 13, 2009
(86) PCT No.: PCT/US09/60521

§ 371 Date: Oct. 10, 2011

Related U.S. Application Data
(60) Provisional application No. 61/105,715, filed on Oct. 15, 2008.

(51) Int. Cl. A61K 9/14; A61K 31/436; C08G 69/48; C08G 69/10; A61K 31/337; C08G 69/08; B82Y 5/00

(52) U.S. Cl. 424/489; 514/449; 514/291; 528/310; 525/432; 525/434; 528/328; 977/773; 977/906

(57) ABSTRACT

The invention provides sequential poly(ester amide)s derived from Proline and that are synthesized by a two-step method, involving a final thermal polyesterification reaction. Molecular weights of polymers prepared by this method are from 14,000 Da to about 77,000 Da.1 When invention proline-based PEAs were thermally characterized, their glass transition temperatures were lower than other alpha-amino acid based poly(ester amides) due to lack of internal hydrogen bonding. These Proline-based PEAs assemble as nano-particles in aqueous solutions and form complexes with various cations and biologies, including hydrophobic small molecule drugs and biologies. Therefore the invention Proline-based PEAs are useful for drug delivery applications requiring a polymer with a molecular weight in the range from 14,000 Da to about 77,000 Da and for fabrication of nanoparticles for delivery of hydrophobic drugs.

Significant inflammatory and immunological challenges face a biomaterial upon implantation or injection such that the historical focus has been on identifying polymers that were permanently biologically inert. However, in many applications, such as the delivery of therapeutic drugs and biologics, fully resorbable polymers are desired. The well-characterized polyesters, e.g. poly(lactic-co-glycolic acid), have been the gold standard for degradable polymers for the past 30 years, but more recently a. new approach utilizing the design and development of enzymatically degradable, protein-like polymers has been promising.

Poly(ester amides) (PEAs) are synthetic, amino acid-based copolymers in which amino acid residues are separated by di-functional hydrocarbon spacers, derived from di-acids and diols. These amino acid-rich polymers possess natural protein-like qualities, resulting in a high capacity for hydrogen bonding between polymer chains and between polymer and a loaded therapeutic, or the polymer and water. The lateral incorporation of a tri-functional amino acid, such as Lysine, Tyrosine or Aspartic acid, within such polymer backbones provides a free carboxylate moiety for subsequent conjugation of therapeutic compounds or other groups providing desired structural or functional properties. In addition, the hydrocarbon spacers endow PEAs with desirable solubility profiles, mechanical properties and processability.

An extraordinarily wide range of mechanical and thermal properties of PEAs can be obtained by judicious incorporation of diol or di-acid units of different lengths and flexibilities (Z. Gomurashvili et al. in: Polymers for Biomedical Applications, A. Mahapatro et al., Eds., American Chemical Society, Washington, D.C. (2008), pp. 10-26). These properties have enabled PEA copolymers to be fabricated into elastomeric coatings, for example for drug eluting stents as well as into micro- and nano-particles for the delivery of a wide range of matrixed therapeutics, including lipophilic drugs and biologic macromolecules. For example, proteins or peptides intended to evoke a protective immune response can be conjugated to the copolymer by formation of amide bonds between free amino groups on the antigen and carboxylate conjugation points of the regular PEA copolymer.

In general, regular PEA polymers can be prepared by interfacial or solution active polycondensation from a diacid chloride (or active di-ester) and a monomer derived from the condensation of the selected diol with two amino acids. However, it is well known that interfacial polycondensation can be difficult to control and optimize because of the large number of factors that needs to be considered. In addition, scaling up and purification of product require precise controls to achieve specific goals, such as optimum yield of linear and high molecular weight polymers.

Reduction in the main-chain hydrogen bonding potential of an amino acid residue is a well established technology: common examples are reversible alcohol-capping of the carbonyl oxygen, reversible capping of the amide nitrogen with a suitable leaving group such as Hmb, or irreversible protection of the amide nitrogen by a methyl group (so called “N-methylation”). Secondary amines are rendered tertiary, and thereby un-reactive, by such capping or protection strategies.

Alone among the 20 common natural amino acids, the amine of Proline is secondary in the free amino acid, and therefore becomes tertiary as the polymerized amino acid residue. Thus Proline has an inherently reduced hydrogen-bonding potential compared with the other 19 common natural amino acids. No derivatization of the free proline amino acid or proline residue is necessary to accomplish this effect.

However, use of Proline as the amino acid incorporated into the backbone of a PEA polymer synthesized using the above-described methods has proven difficult due to decreased reactivity of the secondary amine in Proline as compared with that of the primary amines in such amino acids as Leucine, Glycine, and the like.

Therefore, there is a need in the art for new and better methods of incorporating an amino acid containing a secondary amine, particularly Proline, in fabrication of PEA polymers and for such polymers in which the ring structure in the Proline is not destroyed during fabrication. Moreover, there is a need in the art for new and better biodegradable polymers that chelate metal ions and that, therefore, can be used to complex with biologics for use in a polymer delivery composition.

### SUMMARY OF THE INVENTION

The present invention provides poly(ester amide) (PEA) polymers that are based on L- or D-proline and PEA copolymers containing other hydrophobic alpha-amino acids. In contrast to conventional poly(α-amino acids), the polymers of the present invention possess advantageous aqueous solution behavior and matching defined end groups, which provide binding sites for other chelator groups or macromolecules.

Accordingly in one embodiment, the invention provides biodegradable polymer compositions comprising a PEA polymer having a chemical formula described by general structural formula (I),

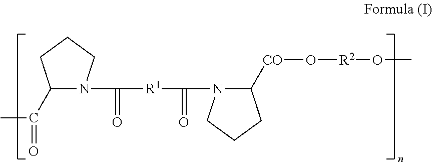

wherein n ranges from about 30 to about 170; R¹ is independently selected from (C₄-C₂₀) alkylene, (C₄-C₂₀) alkenylene or combination thereof; and R² is independently selected from the group consisting of (C₂-C₂₀) alkylene, (C₂-C₂₀) alkenylene, (C₂-C₄) allyloxy (C₂-C₄) alkylene, and combinations thereof, wherein both end groups of the polymer are hydroxyl groups;

or a PEA co-polymer having a chemical formula described by structural formula

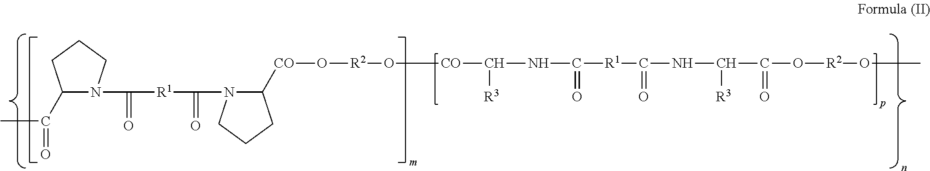

wherein n ranges from about 30 to about 170, m ranges about 0.1 to 0.9; p ranges from about 0.9 to 0.1; R¹ is independently selected from (C₄-C₁₂) alkylene, (C₄-C₁₂) alkenylene, or combination thereof; each R² is independently selected from the group consisting of (C₂-C₁₂) alkylene, (C₂-C₁₂) alkenylene, (C₂-C₄) alkyloxy (C₂-C₄) alkylene, and combinations thereof; the R³s in individual m monomers are independently selected from the group consisting of hydrogen, (C₁-C₆) alkyl, (C₂-C₆) alkenyl, (C₆-C₁₀) aryl (C₁-C₆) alkyl, wherein both end groups of the copolymer are hydroxyl groups.

### A DETAILED DESCRIPTION OF THE INVENTION

The present invention is based on the discoveries that the limitations of achieving sufficient chain length in linear polymers and that the difficulties of purifying diamine monomers containing secondary amines can be overcome utilizing a two-step thermal polyesterification method. In particular, di-p-toluenesulfonic acid salts of bis(L-proline)-α,ω-diol diester can be used in synthesis of Proline-based PEAs. This process is represented schematically in Scheme 1 below:

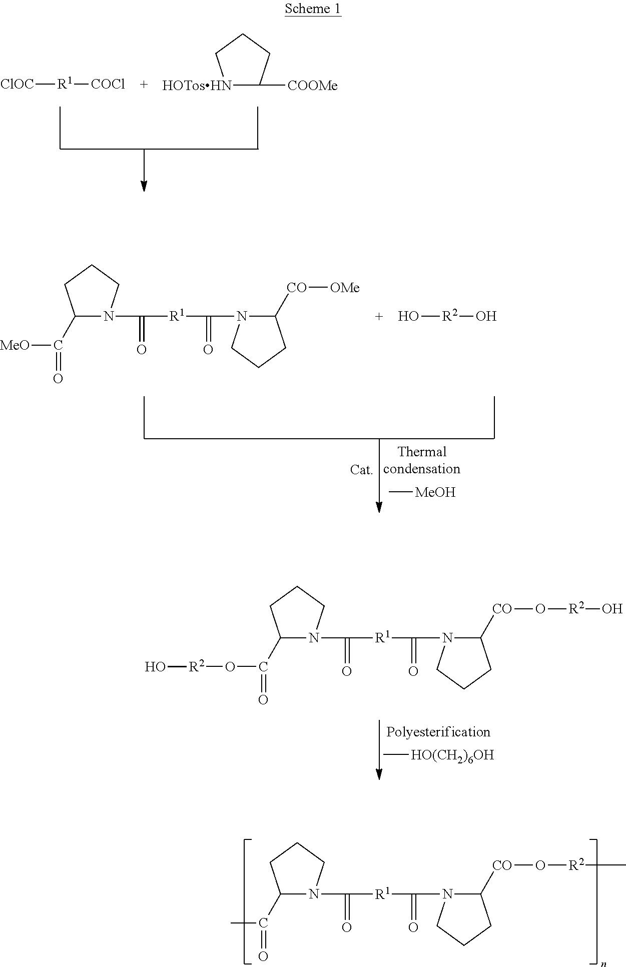

The polyesterification reaction is a melt process and requires high temperatures, between 220° C.-240° C. under vacuum. It is a surprising result of the present invention that the formed PEA polymer and, in particular, the proline ring in the invention Proline-based PEAs will survive the high temperatures required for this high temperature polyesterification reaction.

The present invention provides poly(ester amide) (PEA) polymers that are based on L- or D-proline and copolymers thereof containing other hydrophobic alpha-amino acids. In contrast to conventional poly(α-amino acids), the polymers of the present invention possess advantageous aqueous solution behavior as well as matching defined end groups, which end groups provide binding sites for other chelator groups or macromolecules.

Accordingly in one embodiment, the invention provides biodegradable polymer compositions comprising a PEA polymer having a chemical formula described by general structural formula (I),

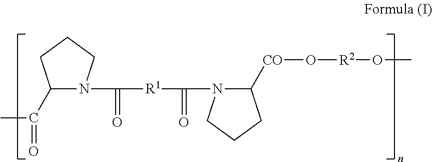

wherein n ranges from about 5 to about 150; R¹ is independently selected from (C₂-C₂₀) alkylene, (C₂-C₂₀) alkenylene or combination thereof; and R² is independently selected from the group consisting of (C₂-C₂₀) alkylene, (C₂-C₂₀) alkenylene, (C₂-C₄) alkyloxy (C₂-C₄) alkylene, and combinations thereof; wherein both end groups of the polymer are hydroxyl groups;

or a PEA co-polymer having a chemical formula described by structural formula (II):

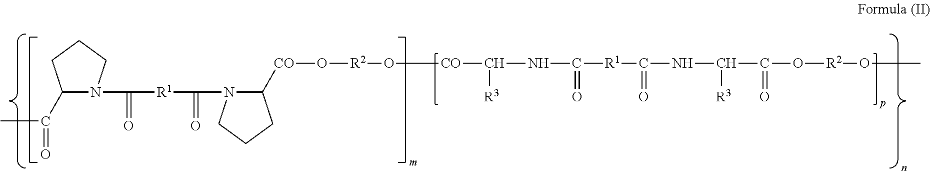

wherein n ranges from about 5 to about 150, m ranges about 0.1 to 0.9; p ranges from about 0.9 to 0.1; R¹ is independently selected from (C₂-C₁₂) alkylene, (C₂-C₁₂) alkenylene, or combination thereof; each R² is independently selected from the group consisting of (C₂-C₁₂) alkylene, (C₂-C₁₂) alkenylene, (C₂-C₄) alkyloxy (C₂-C₄) alkylene, and combinations thereof; the R³s in individual m monomers are independently selected from the group consisting of hydrogen, (C₁-C₆) alkyl, (C₂-C₆) alkenyl, (C₆-C₁₀) aryl (C₁-C₆) alkyl; wherein both end groups of the PEA co-polymer are hydroxyl groups.

It has to be emphasized that the invention methods for preparing PEA polymers and co-polymers using a thermal polyesterification reaction results in linear (i.e., sequential) PEA polymers having a chemical formula described by structural Formulas (I) and (II) in which both end groups of the polymers are hydroxyl groups as shown by Formula (III) below. These hydroxy end-groups readily can be further conjugated with other chelator molecules and with drugs or with macromolecules, such as biologics.

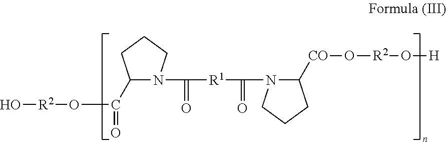

In one embodiment the invention Proline-based PEA polymers have a molecule weight in the range from about 14,000 Da to about 77,000 Da.

As used herein, the term “aryl” in reference to structural formulae herein denotes a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

As used herein, the term “alkenylene” refers to structural formulae herein to mean a divalent branched or unbranched hydrocarbon chain containing at least one unsaturated bond in the main chain or in a side chain.

As used herein, the term “alkenyl” refers to straight or branched chain hydrocarbyl groups having one or more carbon-carbon double bonds.

As used herein, “alkynyl” refers to straight or branched chain hydrocarbyl groups having at least one carbon-carbon triple bond.

As used herein, “aryl” refers to aromatic groups having in the range of 6 up to 14 carbon atoms.

The invention proline-based PEA polymers used in the invention compositions are thermal polyesterification polymers. The ratios “m” and “p” in Formula (II) are defined as irrational numbers in the description of these poly-esterification polymers. Moreover, as “m” and “p” will each take up a range within any poly-esterification polymer, such a range cannot be defined by a pair of integers. Each polymer chain is a string of monomer residues linked together by the rule that all bis(L-proline)-α,ω-diol diester (i) and adirectional amino acid (e.g. Lysine) monomer residues (ii) are linked either to themselves or to each other by a polyamino acid monomer residue (iii). Thus, only linear combinations of i-iii-i; i-iii-ii (or ii-iii-i) and ii-iii-ii are formed. In turn, each of these combinations is linked either to themselves or to each other by a diacid monomer residue (iii). Each polymer chain is therefore a statistical, but non-random, string of monomer residues composed of integer numbers of monomers, i, ii and iii. However, in general, for polymer chains of any practical average molecular weight (i.e., sufficient mean length), the ratios of monomer residues “m” and “p” in formula (II) will not be whole numbers (rational integers). Furthermore, for the esterification of all poly-dispersed copolymer chains, the numbers of monomers i, ii and iii averaged over all of the chains (i.e. normalized to the average chain length) will not be integers. It follows that the ratios can only take irrational values (i.e., any real number that is not a rational number). Irrational numbers, as the term is used herein, are derived from ratios that are not of the form n/j, where n and j are integers.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof. As used herein, the term “adirectional amino acid” means a chemical moiety within the polymer chain obtained from an α-amino acid, such that the R group (for example R⁵ in Formulas II) is inserted within the polymer backbone.

The invention Proline-based PEAs of Formulas I and II contain in the polymer backbone a structure based on the amino acid Proline in which two pendant groups, —(CH₂)₃—, have cyclized to form the chemical structure described by structural formula (IV):

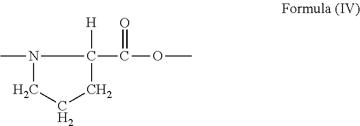

Thus the cyclized pendant groups form an α-imino acid analogous to pyrrolidine-2-carboxylic acid (Proline).

The invention Praline-based polymers can be prepared using a two-step thermal polyesterification reaction outlined in Scheme 2, wherein α,ω, C₂ to C₂₀ diacid chloride, or active di-ester thereof, are contacted with a monomer derived from thermal condensation of two Proline molecules with a C₄ to C₂₀ diol under conditions suitable for a transesterification reaction in aqueous solution containing aprotic solvents, for example at a temperature 220° C.-240° C. under vacuum. The product Proline-based PEA polymer formed by the transesterification reaction is then separated from the aqueous solution using methods known in the art and as described in the Examples herein.

Ester bonds inherent in bis(Proline-acyl)-diester monomers and their derived polymers can be hydrolyzed by bioenzymes, forming non toxic degradation products, including α-amino acids and Proline.

In one alternative, biological α-amino acids in addition to Proline can be used in fabrication of the comonomers used in synthesis of the invention polymers of Formula II. For example, when the R³s in Formula II are CH₂Ph, the biological α-amino acid used in synthesis is L-phenylalanine. In alternatives wherein the R³s are CH₂CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)CH₂CH₃), phenylalanine (when the R³s are CH₂C₆H₅), methionine (when the R³s are —(CH₂)₂SCH₃), L-lysine (wherein R³ is (CH₂)₄NH₂), D- or L-arginine (wherein R³ is (CH₂)₃NHC(═NH)NH₂), L-histidine (wherein R³ is 4-methylene imidazole), aspartic acid (wherein R³ is CH₂COOH), glutamic acid (wherein R³ is (CH₂)₂COOH), and combinations thereof. In yet another alternative embodiment, all of the α-amino acids used in making the invention Proline-based polymers of Formula (II) and compositions thereof are Prolines, wherein the R³s are —(CH₂)₃— and the R³s therein have been cyclized to form the chemical structure described by structural formula (III) as described herein.

In yet another embodiment, the invention provides methods for delivering one or more therapeutic cargo molecules, such as a hydrophobic drug or biologic, to a site in the body of a subject. In this embodiment, the invention methods involve injecting into an in vivo site in the body of the subject an invention composition that has been formulated as a dispersion of polymer nanoparticles wherein at least one cargo molecule is held in encapsulated therein. The injected nanoparticles will slowly release the complexed therapeutic cargo molecules as the composition biodegrades by enzymatic action. The invention nanoparticles can also encapsulate Zn and Ca ions from a buffer solution.

A dispersion of the invention nanoparticles can be injected parenterally, for example subcutaneously, intramuscularly, or into an interior body site, such as an organ. The biodegradable nanoparticles act as a carrier for the at least one, for example two different cargo molecules, into the circulation for targeted and timed release systemically. Invention polymer particles in the size range of about 10 nm to about 500 nm will enter directly into the circulation for such purposes.

The biodegradable polymers used in the invention composition can be designed to tailor the rate of biodegradation of the polymer to result in continuous delivery of the cargo molecule over a selected period of time, depending upon the choice of the building blocks of the polymer, particularly, the amino acids included in the invention composition.

Suitable protecting groups for use in the Proline-based PEA polymers include a tosyl salt (e.g. Tos-OH), or another as is known in the art. Suitable 1,4:3,6-dianhydrohexitols of general formula (III) include those derived from sugar alcohols, such as D-glucitol, D mannitol, or L-iditol. Dianhydrosorbitol is the presently preferred bicyclic fragment of a 1,4:3,6-dianhydrohexitol for use in fabrication of the invention Proline-based polymer delivery compositions.

In one alternative, R³ in Formula II is CH₂Ph and the α-amino acid used in synthesis is L-phenylalanine. In alternatives wherein R³ is CH₂—CH(CH₃)₂, the polymer contains the α-amino acid, leucine. By varying R³, other α-amino acids can also be used, e.g., glycine (when R³ is H), alanine (when R³ is CH₃), valine (when R³ is CH(CH₃)₂), isoleucine (when R³ is CH(CH₃)—CH₂—CH₃), phenylalanine (when R³ is CH₂—C₆H₅), lysine (when R³ is —(CH₂)₄—NH₂); or methionine (when R³ is —(CH₂)₂SCH₃).

The invention Proline-based PEAs are unique because inherent hydrogen bonding, such as is found in other amino acid polymers, is not present. Therefore, the glass transition temperature of these polymers (Tg) is low. Moreover, aqueous solution behavior is unusual. The invention Proline-based polymers form stable nanoparticles in aqueous solution and bind or encapsulate cations and hydrophobic drugs present in the aqueous solution when the nanoparticles precipitate. For example, the presence of Zn²⁺ or Ca²⁺ in a buffer solution can be bound or encapsulated in the polymer nanoparticles precipitated in aqueous solution from the invention polymers.

Moreover, while nanoparticles can be fabricated from other amino acid-based PEA polymers, it has been found that the invention Proline-based PEAs formed by thermal esterification, such as the 8-Pro(6) polymer described in Examples 2 and 3 herein, provides significantly improved incorporation efficiency of hydrophobic drugs. For example, attempts to fabricate docetaxel nanoparticles when regular PEAs that do not contain Proline as in-line amino acids were substituted in place of invention polymers, but regular PEAs of formula Va (PEA I.Ac.H) and Vb (PEA-IV.H), resulted in <30% recovery of docetaxel from aqueous solution, which is considerably lower than the ˜80% obtained with 8-Pro(6) as described in Example 2 herein.

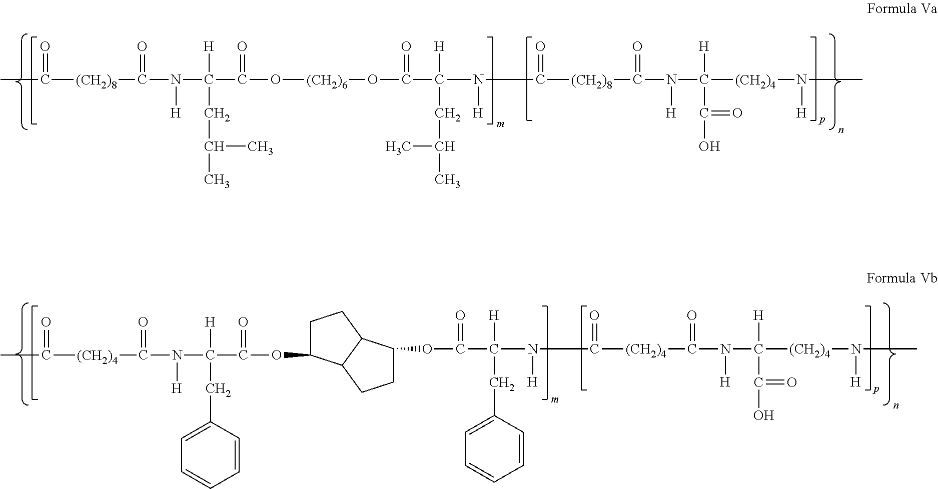

wherein, m=0.75, p=0.25, n=15-45;

In invention polymers, which comprise a bis-L-Proline-containing diol diester monomer, the choice of the in-line α-amino acids (including selection of R³s in Formula II) and the diol used in fabrication of the polymer aid in determination of the electronic properties of the invention Proline-based polymer. For example, the resulting polymer can be water soluble. Chelation of cations at a mol fraction of 1:1 (cation:Proline) neutralizes the in-line imine groups and so the cation-bound polymer becomes a string of alternating hydrophobic segments and neutral polar segments. The resulting cation-bound polymer readily condenses into nanoparticles in buffered aqueous solution.

The following Examples are meant to illustrate and not to limit the invention.

### Example 1

### Product Characterization

The chemical structures of monomers and polymers were characterized by standard chemical methods; NMR spectra were recorded by a Bruker AMX-500 spectrometer (Numega R. Labs Inc. San Diego, Calif.) operating at 500 MHz for ¹H NMR spectroscopy. Solvents CDCl₃ or DMSO-d₆ (Cambridge Isotope Laboratories, Inc., Andover, Mass.) were used with tetramethylsilane (TMS) as internal standard.

Melting points of synthesized monomers were determined on an automatic Mettler-Toledo FP62 Melting Point Apparatus (Columbus, Ohio). Thermal properties of synthesized monomers and polymers were characterized on differential scanning calorimeter (DSC) Mettler-Toledo DSC 822e. Samples were placed in aluminum pans. Measurements were carried out at a scanning rate of 10° C./min under nitrogen flow.

The number and weight average molecular weights (Mw and Mn) and molecular weight distribution (Mw/Mn) of synthesized polymer was determined by Model 515 gel permeation chromatography (Waters Associates Inc. Milford, Mass.) equipped with a high pressure liquid chromatographic pump, a Waters 2414 refractory index detector. 0.1% of LiCl solution in N,N-dimethylacetamide (DMAc) was used as eluent (1.0 mL/min). Two Styragel® HR 5E DMF type columns (Waters) were connected and calibrated with polystyrene standards. Mass Spectra of low molecular weight fractions of polymers were measured on Applied Biosystems Voyager DE Maldi-TOF instrument (Scripps Center of Mass Spectroscopy, San Diego, Calif.). As matrix 2′,4′,6′-trihydroxyacetophenone (THAP) or 3-indole was used.

The particle sizes and zeta potentials were determined on a dynamic light scatter Zetananosizer (Malvern Instruments, UK).

### Monomer Synthesis

Di-p-toluenesulfonic acid salts of bis(L-proline)-α,ω-diol diester, Formula VI;

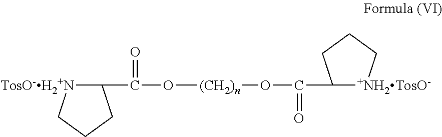

Esterification reactions of L-Proline with aliphatic diols were conducted using a procedure analogous to that described previously for α-amino acids (R. Katsarava et al. J. Polym. Sci. Part A: Polym. Chem. (1999) 37:391-407).

A) Synthesis of Di-p-toluenesulfonic acid salt of bis(L-proline)-1,6-hexanediol diester, (n=6, formula 4). A three-necked, round-bottom flask equipped with a Drierite® drying tube, a Dean-Stark condenser and an overhead stirrer, was charged with the 1,6-hexanediol (17.8 g, 0.152 mol), L-Proline (36.81 g, 0.32 mol), p-toluenesulfonic acid monohydrate (64 g, 0.335 mol), and toluene (1.5 L). The reaction mixture was refluxed for 24 hrs until no more water was distilled. Then it was cooled to room temperature, a toluene layer was decanted off and the oily layer was rinsed with 200 mL of ether, and dried under vacuum. Viscous monomer was then re-dissolved in isopropanol (1:1, w/w) and poured into 3 L of ether. Finally, a hygroscopic product was dissolved in water, and dried on a lyophilizer, followed by drying in a vacuum oven with P₂O₅. Yield: 62%, MS: C₃₀H₄₄N₂O₁₀S_{2 [}656.2]; (-Q1): 655.7. ¹H NMR (D₂O): δ 7.66 (d, 4H, Ar), 7.30 (d, 4H, Ar), 4.39 (t, 2H, ═NH₂⁺—CH—CO), 4.17 (m, 4H, CO—O—CH₂—), 3.37 (m, 4H, ═NH₂⁺—CH₂—CH₂—), 2.36-2.07 (m,m, 4H, NH—CH—CH₂—), 2.33 (s, 6H, Me), 1.99 (m, 4H, ═N—CH₂—CH₂—CH₂), 1.59 (m, 4H, —O—CH₂CH₂), 1.29 (t, 4H, —O—CH₂—CH₂—CH₂—).

B) Synthesis of Di-p-toluenesulfonic acid salt of bis-(L-proline)-1,3-propanediol diester, (n=3, formula x) was prepared using a procedure analogous to that described in A) above. Hygroscopic white crystalline material was recovered in 98% yield; ¹H NMR (D₂O): δ 7.68 (d, 4H, Ar), 7.36 (d, 4H, Ar), 4.48 (t, 2H, ═NH₂⁺—CH—CO), 4.33 (m, 4H, CO—O—CH₂—), 3.41 (m, 4H, ═NH₂⁺—CH₂—CH₂—), 2.43-2.15 (m,m, 4H, NH—CH—CH₂—), 2.38 (s, 6H, Me), 2.08 (m, 2H, —O—CH₂CH₂CH₂—), 2.05 (m, 4H, ═NH₂⁺—CH₂—CH₂—CH₂).

### Synthesis of Active Di-Esters of Aliphatic Dicarboxylic Acids for Solution Polycondensation (Compound 2 in Scheme 1)

Active ester di-oxysuccinimidyl sebacate was prepared as described previously (R. D. Katsarava et al. Synthesis of Polyamides Using Activated bis-oxysuccinimide esters of dicarboxylic acid. Vysocomol. Soed. A (1984) 27(7):1489-1497).

A) Synthesis of di-pentafluorophenyl sebacate: To the chilled (0° C.) solution of 21.7 g (0.118 mol) of pentafluorophenol and 16.43 mL (0.118 mol) of triethylamine in 120 mL of ethylacetate, a solution of sebacoyl chloride 12 mL (0.056 mol) was added dropwise over 30 minutes. Afterwards, the reaction mixture was warmed up to room temperature (Lt.), stirred for 8 hours and filtered. Ethylacetate solution was evaporated and the obtained solid product was washed with ether and dried. Yield after recrystallization in acetone was 10.7 g, Mp=62.6° C. ¹H NMR (DMSO-d₆): δ 2.77 (t, 4H), 1.67 (q, 4H) 1.38-1.32 (m, 8H).

Elemental Analysis (Elem. Anal.) Calcd. for C₂₂H₁₆F₁₀O₄ (534.34): C, 49.45; H, 3.02. Found: C, 49.21H, 2.57.

### Synthesis of Monomers for Thermal Polyesterification:

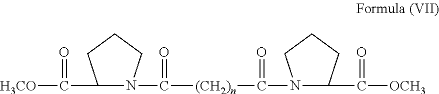

Synthesis of Di-methyl ester of bis-(L-prolyl)-sebacamide, (Formula VII, n=8). A 250 mL round-bottom flask equipped with an addition funnel and a magnetic stirrer was purged with Argon gas and charged with L-proline methyl ester hydrochloride 8.62 g (66.7 mmol), triethylamine 19 mL (0.136 mol), and 40 mL of chloroform and placed on ice-bath. Then 7.09 mL (33.2 mmol) of sebacoyl chloride was diluted in 8 mL of chloroform and slowly added for 45 min so that the reaction temperature was kept at <8° C. The reaction was continued for additional 12 h at room temperature. Chloroform solution was extracted with 100 mL of water, then with brine 2×100 mL, and with anhydrous Na₂SO₄, filtered, and evaporated under reduced pressure. The resulting viscous liquid was purified on a silica column using Ethylacetate/Hexanes 4:6 v/v and then 8:2 v/v. Pale yellow crystals were formed after standing in a refrigerator over 2-3 days, with final yield of 7.91 g (56%); M.p. 44.7° C. (DSC, 2°/min), ¹H NMR (DMSO-d₆): δ 4.26 (dd, 2H, ═N—CH—CO), 3.59 (s, 6H, Me), 3.49 (m, 4H, ═N—CH₂—CH—), 2.25 (m, 4H, CO—CH₂—CH₂), 2.14-1.80 (m,m, 4H, NH—CH—CH₂—), 1.89 (m, 4H, ═N—CH₂—CH₂—CH₂), 1.46 (m, 4H, CH₂CH₂CO), 1.25 (m, 8H, CH₂CH₂CH₂CO).

Synthesis of Di-methyl ester of bis-(L-prolyl)-adipamide, (Formula 5, n=4): Synthesis was carried out in chloroform analogous to a procedure previously described, using adipoyl chloride. Formed orange-colored oil was purified on a column, using hexanes/ethylacetate eluent, changing from 6:4 v/v to 2:8 v/v. Yellow crystals were formed after 3-4 days storage in a refrigerator; Mp=62° C., (DSC, 2° C./min). Product yield was between 60-67 (DMSO-d₆): δ 4.26 (q, 2H, ═N—CH—CO), 3.59 (s, 6H, Me), 3.50 (m, 4H, ═N—CH₂—CH—), 2.27 (m, 4H, CO—CH₂—CH₂), 2.15-1.81 (m,m, 4H, NH—CH—CH₂—), 1.89 (m, 4H, ═N—CH₂—CH₂—CH₂), 1.51 (m, 4H, CH₂CH₂CO). El. Anal. Calcd. for C₁₈H₂₈N₂O₆ (368.19) C, 58.68; H, 7.66; N, 7.60. Found C, 58.52; H, 7.71.

### Polymerization

### Interfacial Polyamidation

A) PEA 8-Pro(6) synthesis; (Formula I, R¹=(CH₂)₈, R²—(CH₂)₆, 5.0 g scale): In 21.8 mL aqueous 0.32 M solution of sodium carbonate, were dissolved 4.57 g (6.966 mmol) of diester-diamine (n=6, formula 4). Once dissolved, formed monomer solution was added into a homogenizer and 1.489 mL (6.96 mmol) sebacoyl chloride solution in 14 mL dichloromethane (DCM) was added. The use of excess base (8 eq) caused the reaction not to stir homogenously. Additional 5 mL of DCM and 5 mL of water were added and the solution was stirred for a total of 30 mins. Afterwards the organic layer was extracted with acetic acid. This extraction was repeated until the DCM layer was clear and transparent. The organic layer was then dried over Na₂SO₄ and filtered through qualitative filter paper. The polymer solution was then concentrated. Weight average Mw of the crude product was 13,481 Da with a polydispersity of 1.046. The polymer was then concentrated to dryness and further dried in vacuum oven over the weekend. Recovered yield was 1.96 g, (30.7%).

Polymer Synthesis, 8-Pro(6) (5.0 g scale) with prolonged dichloride addition: A procedure analogous to that described in A) above was used, except that the solution was stirred for a total of 50 mins, with 0.5 mL of sebacoyl chloride being added at 10 minute intervals for the first 30 minutes. Afterwards the organic layer was extracted with acetic acid to remove excess sodium carbonate from the organic layer. This extraction was repeated until cloudiness in the DCM was removed, yielding a clear and transparent organic layer, which was then dried over Na₂SO₄ and filtered thru qualitative filter paper. The polymer solution was then concentrated to remove excess DCM. Then the polymer was dissolved in H₂O and placed into a dialysis bag for further purification over a weekend. Weight average Mw of the crude product was 20060 with a polydispersity of 1.171.

### Solution Active Polycondensation

A polycondensation reaction was conducted between diamine monomers of Formula 4 and active esters of sebacic acid.

General procedure: To the stirred mixture of 6 mmol of compound 1 above and 6 mmol of compound 2 above in 4.12 mL of N,N-dimethyl formamide (DMF), 0.88 mL (6.3 mmol) of triethylamine (total volume 5.0 mL, c=1.2 mol/L) was added under dry nitrogen and heated at 65° C. for 48 hours. In all cases, the reaction proceeded homogeneously. Crude molecular weight of formed polymer was determined by GPC. The obtained viscous reaction solution was poured into iced water and the precipitated product was filtered off and thoroughly washed with water. The obtained solid products were dried at 40° C. in vacuo.

TABLE 1
Solution polycondensation of L-Pro(6)•TosOH (n =
6, formula 4) monomer with sebacic acid derivatives.

| Di-acid | Reaction | Reaction | Mw ^{a)} | Mn ^{a)} |
|---|---|---|---|---|
| derivative | Conditions | Time [h] | [g/mol] | [g/mol] |
| Di-penta- | 45°→ 65° C.; | 48 | 15 000 | 13 000 |
| fluorophenyl | TEA, DMF ^{b)} | | | |
| sebacate | | | | |
| Di-oxy- | 45°→ 65° C.; | 48 | Polymer not | — |
| succinimidyl | TEA, DMF | | detected | |
| sebacate | | | | |

^{a)} from GPC measurement, eluent N,N-dimethylacetamide
^{b)} TEA = triethanolamine; DMF = dimethylformamide

As can be seen from the results summarized in Table 1, low temperature solution polycondensation of L-Pro(6). TosOH (n=6, formula 4) monomer with sebacic acid derivatives yielded either polymer of low Mw and Mn (when the di-acid derivative was Di-penta-fluorophenyl sebacate) or no polymer at all (when the di-acid derivative was Di-oxy-fluorophenyl sebacate).

### Thermal Polyesterification

To illustrate the process and properties of Proline-based PEAs synthesized by thermal polyesterification the following experiment was conducted to fabricate PEA-8-Pro(6), (Formula I, where (R¹=C₈ alkylene and R²=C₆ alkylene, n=110-160).

A 250 mL three-neck round-bottom flask equipped with a magnetic stirrer and argon in- and outlet was charged with 2.59 g (22 mmol, 2.3 eq.) of 1,6-hexanediol, 4.05 g (9.54 mmol) of di-methyl ester of bis-(L-prolyl)-sebacamide, (Formula 5, n=8) and 32 uL titanium butoxyde (0.095 mmol, 0.01 eq). The flask was heated in oil bath at 160° C. to 190° C. under slow flow of argon for 2.5 h. Then the argon outlet was closed and a vacuum pump (0.5 mm Hg) was attached while the temperature of the bath was raised to 225° C. To improve evacuation of the diol, the reaction was periodically stopped (every 3 h) by cooling it to room temperature, and then the diol that had condensed on the flask wall was removed. Progress of polymerization was monitored by gel permeation chromatography (GPC).

The reaction was prolonged until no more diol was distilled off (8 h). Formed polymer then was dissolved in 15 mL of chloroform and precipitated into 200 mL ethylacetate/ether 1:1 v/v. Viscous oily product was collected, re-dissolved in 20 mL of methanol, filtered through 0.45 μm PTFE filter, then cast on a Teflon tray and dried under vacuum. Yield was 3.69 g (81%); Tg=5° C. (DSC, 10°/min).

### Example 2

### Synthesis of PEA 8-Pro(6) Polymers with Metal Chelator End Groups

Covalent attachment of metal chelating molecules to the hydroxyl end groups of invention polymer changes the binding capacity of the invention PEA polymer with various cations (e.g., Zn²⁺, Ni²⁺, Ca²⁺). These formulations with metal chelated end groups will bind to various biologics containing metal-binding amino acids, for example His-tagged proteins. The group of metal-chelating molecules can be used to end-cap the invention polymers include, for example, imidoacetic acid, for example: Ethylenediaminetetraacetic acid (EDTA), Diethylenetriaminepentaacetic acid (DTPA), and Ethylene glycol-bis(2-aminoethylether)-N,N,N′,N′-tetraacetic acid (EGTA).

EDTA binding to PEA 8-Pro(6) polymer was accomplished as illustrated in Scheme 3 below:

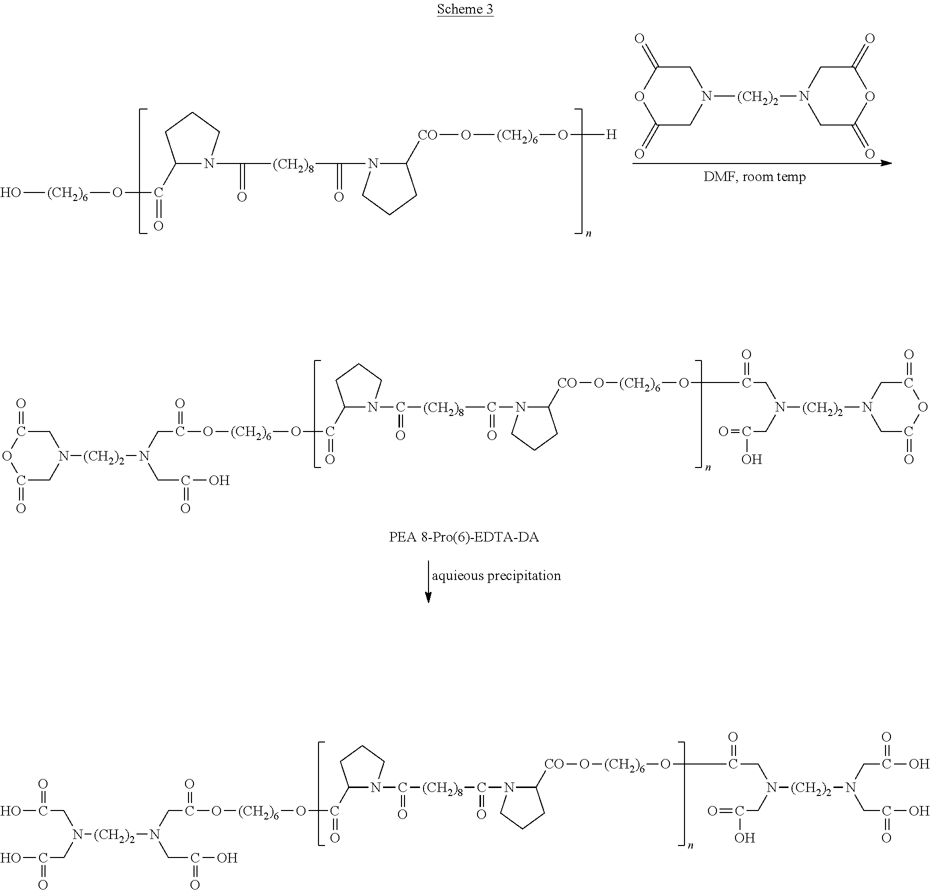

PEA 8-Pro(6)-EDTA (5 g scale): In 40 mL vial, 5.1 g of PEA 8-Pro(6) (Mw=28,000 Da) was dissolved in 15 mL NDN-dimethylformamide (DMF), under argon. Once dissolved, 49 ul (1 eq) TEA was added to the solution. In a separate 40 mL vial, 10 mL DMF was added to 0.9066 g (10 eq) EDTA-Dianhydride (Aldrich). The polymer solution was added to EDTA-DA while stirring, the reaction was purged with argon and stirring was continued overnight at room temperature. Then the reaction was heated at 45° C. for 1 hour and precipitated in 200 mL distilled water. The water was decanted, polymer was re-dissolved in 20 mL of methanol and precipitated in 100 mL of water containing 3 g of CaCl₂. The polymer crashed out as a white sticky solid and the supernatant, which was initially cloudy, turned clear after about 30 minutes of stirring. The precipitate was rinsed with deionized water, redissolved in MeOH, filtered through 1.0 um PTFE filters into a Teflon tray, and dried in the oven at 65° C. Mw=32,000 Da. Yield was 2.3 g. Maldi-TOF MS and ¹H-NMR spectrums of the end-capped polymer has confirmed the presence of EDTA ends.

PEA 8-Pro(6)-EDTA-DA intermediate product from scheme 3, with active di-anhydride end groups, can be further conjugated in-situ with another hydrophilic polymer, for example, polysaccharides and polyethyleneglycols: mPEG-OH or mPEG-NH₂, forming metal-chelating ABA block co-polymers, as shown in scheme 4.

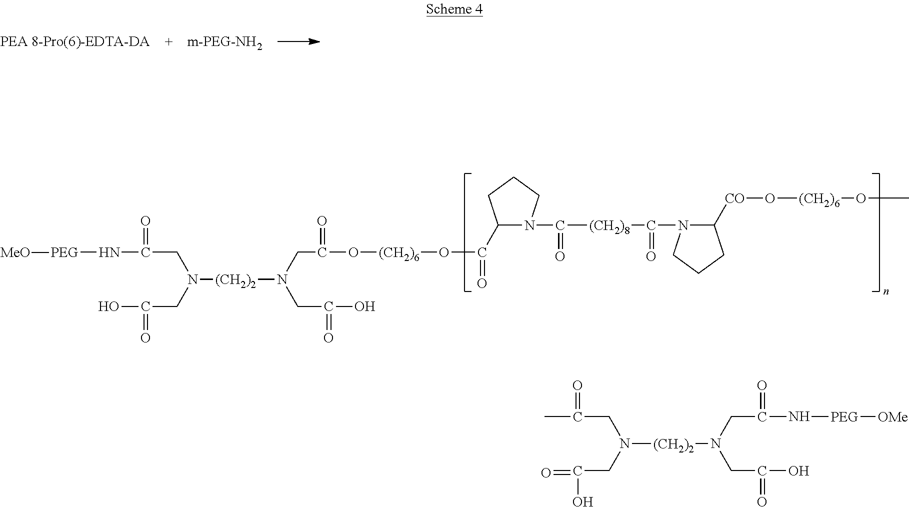

Alternatively, invention PEA 8-Pro(6) polymer can be first covalently bound with PEG-diol via a succinic acid linker, which further can be end-capped with a chelator molecule, as shown in scheme 5:

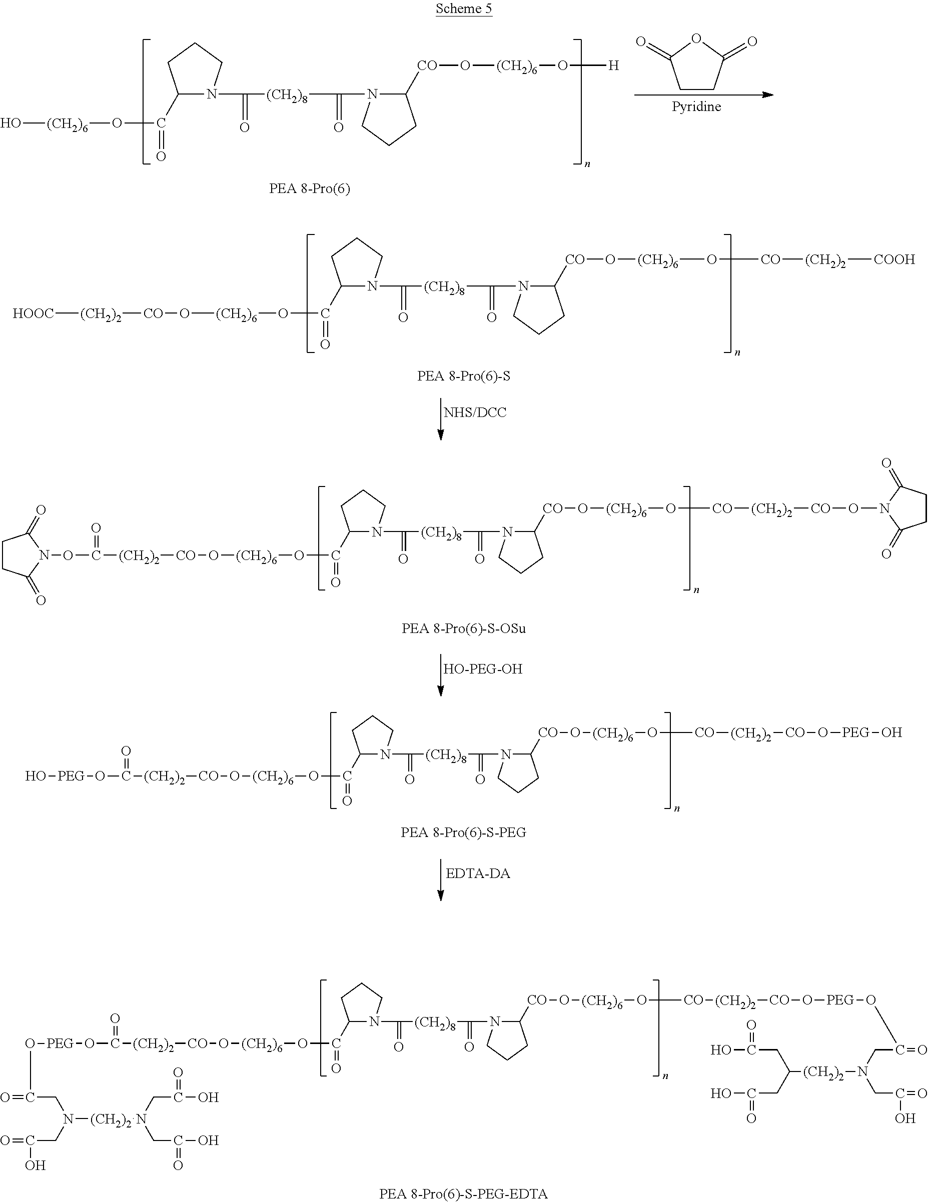

### Example 3

### Preparation of Docetaxel Nanoparticles

In 1.00 mL of ethanol, 4.29 mg of docetaxel and 10.0 mg of PEA-8-Pro(6), (Formula I, where (R¹=(CH₂)₈ and R²=(CH₂)₆, n=110-160), were co-dissolved. The docetaxel/polymer solution was added slowly to 9.00 mL of a stirred aqueous buffer (in this case citrate, pH 7.0) containing 0.1% Bovine Serum Albumin (BSA), resulting in formation of nanoparticles by precipitation. The translucent dispersion of nanoparticles was transferred to regenerated cellulose dialysis tubing (MWCO 3500 Da) and dialyzed against aqueous buffer (100×v/v) at room temperature for 16 h to remove residual ethanol. The typical diameter of the docetaxel/polymer particles was 200-240 nm (PDI<0.15) with a zeta potential of −17 to −21 mV (determined on Malvern Zetasizer). A control formulation in which the invention PEA polymer was omitted during fabrication of particles, yielded only micron-scale crystals.

After processing, 77% of the docetaxel and 70% of the polymer were recovered, based on RP-HPLC and Amino Acid Analysis (AAA), respectively. Less than 8% of the docetaxel and polymer were removed after filtration with a 1 μm filter, demonstrating that the formulation was substantially sub-micron. However, no docetaxel was detected after filtration in the control formulation prepared without polymer.

Final loading of hydrophobic drug into 8-Pro(6) nanoparticles formed by microprecipitation was calculated as the mass of drug (API) divided by the sum of the polymer and drug mass, i.e. (API)/(Polymer+API). Using this formula, loading of docetaxel was calculated to be 31%.

### Preparation of Rapamycin Nanoparticles

In 0.700 mL of dimethyl sulfoxide DMSO, 1.25 mg of rapamycin and 5.0 mg of PEA-8-Pro(6), (Formula I, where (R¹=(CH₂)₈ and R²=(CH₂)₆, n=110-160), were co-dissolved. The rapamycin/polymer solution was added slowly to 9.30 mL of a stirred aqueous buffer (e.g. HEPES, pH 7.0), resulting in formation of nanoparticles. The translucent dispersion was transferred to regenerated cellulose dialysis tubing (MWCO 3500 Da) and dialyzed against aqueous buffer (100×v/v) at room temperature for 16 h to remove residual DMSO. The diameter of the rapamycin/polymer particles was 106 nm (PDI<0.10) with a zeta potential of −41 mV (Malvern Zetasizer). In contrast, micron-scale particulate was obtained when the PEA was omitted during fabrication. After filtration using a 5 μm filter, 72% of the rapamycin was recovered in the polymer formulation based on RP-HPLC, whereas 6% was recovered in the polymer-free control. Final loading of hydrophobic drug Rapamycin into 8-Pro(6) nanoparticles formed by microprecipitation was calculated to be 20% using the formula described in Example 2 above.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

That which is claimed is:

1. A composition comprising at least one biodegradable poly(ester amide) (PEA) polymer having a chemical formula described by general structural formula (I), 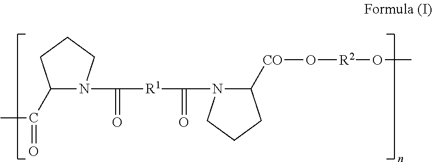
wherein n ranges from about 30 to about 170; R¹ is independently selected from (C₄-C₂₀) alkylene, (C₄-C₂₀) alkenylene or combination thereof; and R² is independently selected from the group consisting of (C₂-C₂₀) alkylene, (C₂-C₂₀) alkenylene, (C₂-C₄) alkyloxy (C₂-C₄) alkylene, and combinations thereof, wherein both end groups of the polymer are hydroxyl groups;
or a PEA co-polymer having a chemical formula described by structural formula 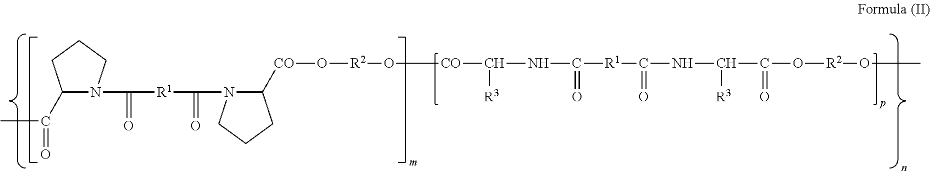
wherein n ranges from about 30 to about 170, m ranges about 0.1 to 0.9; p ranges from about 0.9 to 0.1; R¹ is independently selected from (C₄-C₁₂) alkylene, (C₄-C₁₂) alkenylene, or combination thereof; each R² is independently selected from the group consisting of (C₂-C₁₂) alkylene, (C₂-C₁₂) alkenylene, (C₂-C₄) alkyloxy (C₂-C₄) alkylene, and combinations thereof; the R³s in individual m monomers are independently selected from the group consisting of hydrogen, (C₁-C₆) alkyl, (C₂-C₆) alkenyl, (C₆-C₁₀) aryl (C₁-C₆) alkyl, and wherein both end groups of the copolymer are hydroxyl groups.

2. The composition of claim 1, wherein the R¹s are independently selected from (C₆-C₈) alkylene.

3. The composition of claim 1, wherein the average molecular weight (Mw) of the PEA polymer is in the range from about 14,000 Da to about 77,000 Da.

4. The composition of claim 1, wherein the PEA polymer complexes Zn²⁺ and Ca²⁺ in a buffer solution.

5. The composition of claim 1, wherein the composition is fabricated as nanoparticles.

6. The composition of claim 1, further comprising a hydrophobic drug and the composition microprecipitates in aqueous solution as nanoparticles that encapsulate the hydrophobic drug.

7. The composition of claim 1, wherein the PEA polymer is described by Formula (I) wherein R¹ is (C)₈ alkylene, R² is (C)₆ alkylene, and n is from 110 to 150.

8. The composition of claim 1, wherein the nanoparticles encapsulate Zn²⁺ and Ca²⁺ in a buffer solution.

9. The composition of claim 8, wherein the end groups of the polymer have been reacted with Ethylenediaminetetraacetic acid to end-cap the polymer.

10. The composition of claim 9, wherein the end-capped polymer is additionally reacted with Poly(ethylene glycol) polymer to form a metal-chelating ABA-triblock polymer.

11. The composition of claim 6, wherein the hydrophobic drug is docetaxel at from 30 to 40 weight % or rapamycin at 20 to 30 weight % of the nanoparticles.

12. A method for administering a hydrophobic drug to a subject comprising encapsulating the hydrophobic drug in nanoparticles of the PEA polymer of claim 6 and administering the nanoparticles to the subject.

13. A method for synthesizing the at least one PEA polymer of claim 1, said method comprising:
contacting α,ω C₂ to C₂₀ diacid chloride, or active di-ester thereof, and a monomer derived from thermal condensation of a C₄ to C₂₀ diol with two Proline molecules under conditions suitable for a transesterification reaction in aqueous solution, and separating the PEA polymer formed by the transesterification reaction from the aqueous solution.

14. The method of claim 13, wherein the conditions for the transesterification reaction comprise a temperature in the range from about 220° C. to about 240° C. under vacuum.

15. The method of claim 13, wherein the diol is HO(CH₂)₆₋₈OH.

16. The method of claim 13, wherein the average molecular weight (Mw) of the PEA polymer formed is in the range from about 14,000 Da to about 77,000 Da.
